(19) United States
(12) Patent Application Publication
(10) Pub. No.: US 2009/0258000 A1
(43) Pub. Date: Oct. 15, 2009

(54) MUCOSALLY NON-IRRITATIVE AMPHOTERICIN B FORMULATIONS AND METHODS FOR TREATING NON-INVASIVE FUNGUS-INDUCED MUCOSITIS

(75) Inventors: FRANCIS E. O'DONNELL, JR., Longboat Key, FL (US); Donald Deroo, Newton, CT (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: Accentia Biopharmaceuticals, Inc., Tampa, FL (US)

(21) Appl. No.: 12/415,857

(22) Filed: Mar. 31, 2009

Related U.S. Application Data
(63) Continuation of application No. PCT/US2007/080333, filed on Oct. 3, 2007.
(60) Provisional application No. 60/881,270, filed on Jan. 19, 2007.
(60) Provisional application No. 60/849,028, filed on Oct. 3, 2006.

(51) Int. Cl. A61K 38/47; A61K 31/7048

(52) U.S. Cl. 424/94.61; 514/31

(57) ABSTRACT

The present invention is directed to compositions and methods for non-irritatively treating and preventing non-invasive fungus-induced mucositis. Specifically, the invention involves compositions including a mucosally non-irritative mixture of amphotericin B and a pharmaceutically acceptable carrier. Such compositions can be non-irritatively mucoadministered to prevent, reduce, or eliminate chronic non-invasive fungus-induced mucositis conditions.

### RELATED APPLICATIONS

This application is a continuation of International Application No. PCT/US2007/080333, filed Oct. 3, 2007, which claims the benefit of U.S. Provisional Application No. 60/849,028, filed Oct. 3, 2006 and U.S. Provisional Application No. 60/881,270, filed Jan. 19, 2007, each of which is hereby incorporated by reference herein in its entirety, including any figures, tables, nucleic acid sequences, amino acid sequences, or drawings.

### TECHNICAL FIELD

The present invention relates to compositions and methods for the non-irritative treatment and prevention of non-invasive fungus-induced mucositis.

### BACKGROUND

Mucositis, the inflammation of mucosal tissue, is a serious medical problem that affects millions of people worldwide. For example, conservative estimates indicate that between 20 and 40 million Americans suffer from chronic rhinosinusitis, an inflammation of the nasal cavity and/or paranasal sinuses.

U.S. Pat. Nos. 6,555,566, 6,291,500 and 6,207,703, by Dr. Jens Ponikau and assigned to the Mayo Foundation For Medical Education And Research, describes and claims methods of treating non-invasive fungus-induced rhinosinusitus, asthma, or intestinal mucositis by directly mucoadministering to at least a portion of the nasal-paranasal anatomy of the subject a formulation including an antifungal in an amount, at a frequency, and for a duration effective to reduce or eliminate the non-invasive fungus-induced rhinosinusitus, asthma, or intestinal mucositis. The contents of these references are incorporated in their entireties by this reference.

### SUMMARY

The present invention provides universally non-irritative compositions for mucoadministration. The present invention is based, at least in part, on compositions and methods which cause no or negligible burning when mucoadministered. The present invention provides mucosally non-irritative compositions and methods useful for treating mucositis. Such compositions will allow for the mucoadministration of amphotericin B to a larger, more comprehensive population of subjects than the population that currently tolerates conventional formulations. Accordingly, in one aspect the present invention provides a composition for mucoadministration. The composition generally includes a mucosally non-irritative mixture of amphotericin B and a pharmaceutically acceptable carrier, e.g., a universally mucosally non-irritative mixture.

In some embodiments, the pharmaceutically acceptable carrier includes sodium phosphate dibasic and sodium phosphate monobasic.

In some embodiments, the composition includes amphotericin B in an amount of between about 0.27% and about 0.50% by weight of the total composition. In other embodiments, the composition also includes water. In embodiments where the composition includes water, the amphotericin B can be present in an amount of about 0.01% by weight of the total composition. In some embodiments, the pharmaceutically acceptable carrier includes sodium phosphate dibasic, sodium phosphate monobasic and water.

In some aspects, the present invention provides a composition for mucoadministration which includes between about 0.27% and about 0.50% by weight amphotericin B; between about 45% and about 70% by weight sodium phosphate dibasic; and between about 30% and about 55% by weight sodium phosphate monobasic.

In some embodiments, the composition is a powder. In other embodiments, the composition is suitable for incorporation into a solution or suspension. For example, the composition can be in a solid form, wherein the solid form is suitable for incorporation into a solution or suspension.

In some aspects, the present invention provides a composition for mucoadministration which includes amphotericin B; sodium phosphate dibasic; sodium phosphate monobasic; and at least about 96.25% by weight water. For example, the composition can include about 0.01% by weight amphotericin B; about 1.59% by weight sodium phosphate dibasic; about 0.96% by weight sodium phosphate monobasic; and about 97.44% by weight water.

In other aspects, the present invention provides a composition consisting essentially of amphotericin B, sodium phosphate dibasic, sodium phosphate monobasic and water.

In some embodiments, the composition is free or essentially free of propylene glycol. In other embodiments, the composition is free or essentially free of sodium metabisulfate. In still other embodiments, the composition is free or essentially free of carboxymethylcellulose sodium. In other embodiments, the composition is free or essentially free of methylparaben. In yet other embodiments, the composition is free or essentially free of propylparaben. In yet other embodiments, the composition is free or essentially free of desoxycholate, e.g., sodium desoxycholate (also referred to as sodium deoxycholate).

In some embodiments, the compositions of the present invention provide a low maximum plasma concentration upon direct mucoadministration.

In some embodiments, the compositions of the present invention are at least about 90% stable for up to 18 months under a nitrogen atmosphere. In some embodiments, the compositions of the present invention are at least about 95% stable for up to 18 months under a nitrogen atmosphere. In some embodiments, the compositions of the present invention are at least about 20% more stable under a nitrogen atmosphere than under an oxygen atmosphere.

In some aspects, the present invention provides a method of treating a subject having non-invasive fungus-induced mucositis. The method generally includes mucoadministering any composition as described herein. In other aspects, the method includes non-irritatively mucoadministering a composition comprising amphotericin B and a pharmaceutically acceptable carrier.

In some embodiments, the composition is administered in an amount, at a frequency, and for a duration effective to reduce or eliminate the non-invasive fungus-induced mucositis. In some embodiments, the subject is a human.

In some embodiments, the non-invasive fungus-induced mucositis is non-invasive fungus-induced rhinosinusitis, e.g., non-invasive fungus-induced rhinosinusitis with polyp formation or polypoid change and/or chronic non-invasive fungus-induced rhinosinusitis.

In some embodiments, the method includes administering a composition wherein the pharmaceutically acceptable carrier comprises sterile water. In some embodiments, mucoadministration includes irrigating the nasal-paranasal anatomy of the subject with a liquid form of the composition. In other embodiments, mucoadministration includes applying an aerosol form of the composition to the nasal-paranasal anatomy of the subject.

In some embodiments, the composition includes between about 50 μg and about 1000 μg of Amphotericin B per milliliter of sterile water, e.g., between about 100 μg and about 500 μg of Amphotericin B per milliliter of sterile water. In some embodiments, the composition includes about 100 μg of Amphotericin B per milliliter of sterile water. In other embodiments, the effective amount includes about 5 mL to about 100 mL of the composition per nostril of the subject. In still other embodiments, an effective amount comprises about 20 mL of the composition per nostril of the subject. In some embodiments, the compositions of the present invention include about 100 μg of amphotericin B per milliliter of aqueous carrier and about 20 ml of the composition is administered to a subject in each nostril twice daily.

In some embodiments, mucoadministration is achieved via a pump spray. Accordingly, in some embodiments, mucoadministration includes from 1 to 4 pumps per nostril, e.g., 3 pumps per nostril. In some embodiments, the pump dispenses between about 50 μL and about 200 μL of the composition, e.g., about 100 μL of the composition.

In some embodiments, the effective frequency of mucoadministration is from about four times a day to about once every other week. In other embodiments, the effective frequency of mucoadministration is from about three times a day to about once a week. In still other embodiments, the effective frequency of mucoadministration is from about one to four times a day, e.g., three times a day. In still other embodiments, the effective frequency of mucoadministration is more frequent than once a day. In some embodiments, the effective duration is greater than about 30 days. In other embodiments, the effective duration is greater than about 60 days.

In some embodiments, polyposis is improved in the subject. In some embodiments, sinus inflammation is improved in the subject.

In some aspects, the present invention provides a method for reducing eosinophil in a subject. The method can generally include non-irritatively mucoadministering a composition comprising amphotericin B and a pharmaceutically acceptable carrier. In other aspects, the present invention provides a method for reducing the amount of major basic protein in the mucosa of a subject. The method can generally include non-irritatively mucoadministering a composition comprising amphotericin B and a pharmaceutically acceptable carrier.

### BRIEF DESCRIPTION OF THE FIGURES

FIG. 1 is a graph depicting the plasma concentration of exemplary Amphotericin B formulations of the present invention in Gottingen Mini-Pigs.

FIG. 2 is a graph depicting the decrease in endoscopy scores at week 16 after administration of placebo and exemplary Amphotericin B formulations of the present invention.

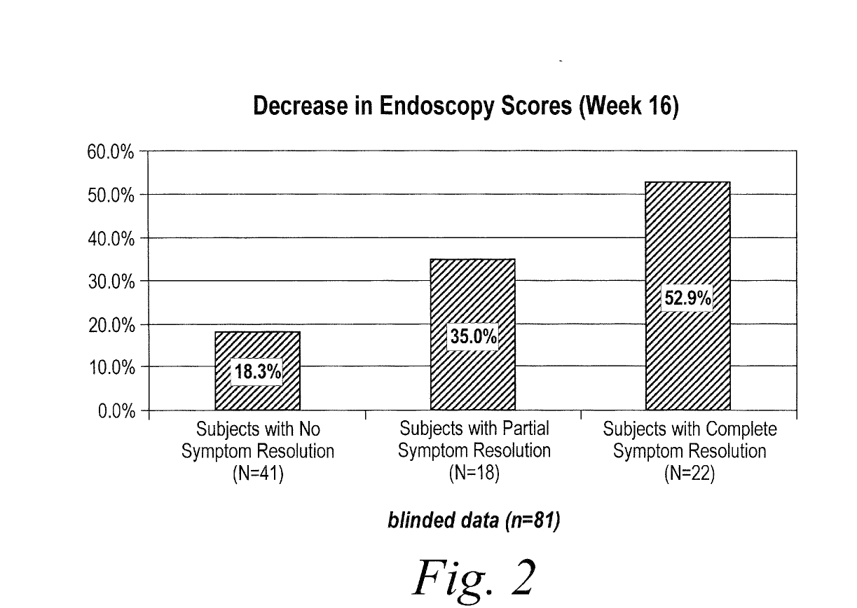

FIG. 3 is a graph depicting the CT scan results after administration of placebo and exemplary Amphotericin B formulations of the present invention.

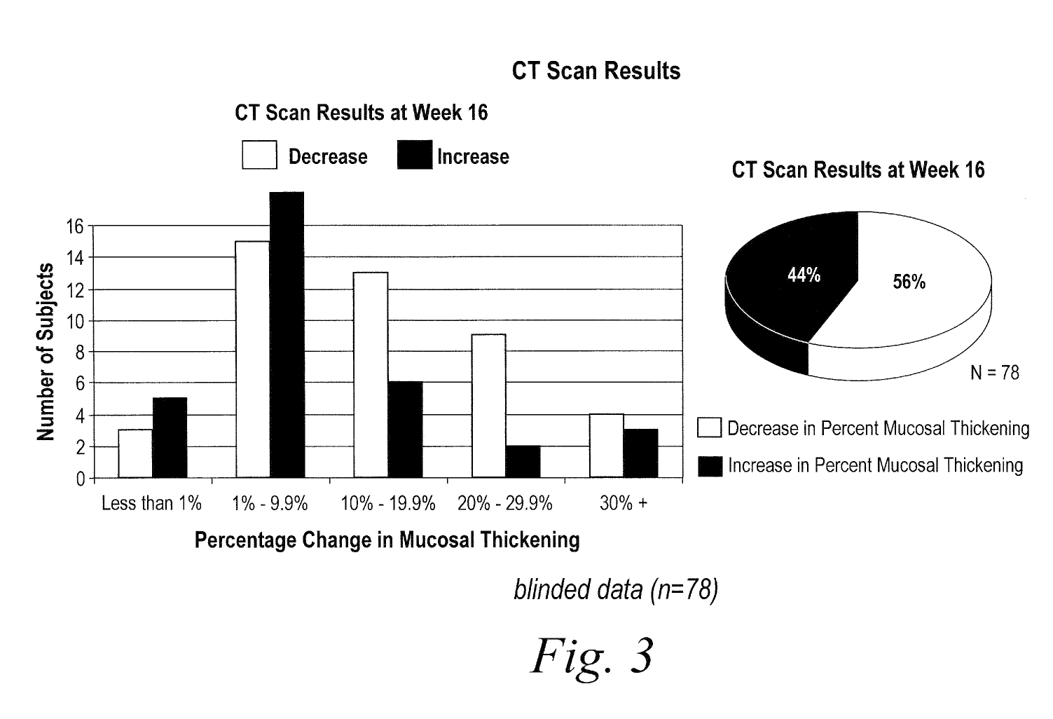

### DETAILED DESCRIPTION

The present invention is directed to methods and compositions for treating and preventing non-invasive fungus-induced mucositis. Specifically, the present invention provides compositions including a mucosally non-irritative mixture of amphotericin B and a pharmaceutically acceptable carrier. Such compositions can be non-irritatively mucoadministered to prevent, reduce, or eliminate chronic non-invasive fungus-induced mucositis conditions.

In order to more clearly and concisely describe the subject matter of the claims, the following definitions are intended to provide guidance as to the meaning of specific terms used herein.

As used herein, the terms “non-irritative” and “non-irritatively” refer to compositions and methods which exhibit no or negligible burning, stinging, itching or otherwise uncomfortable sensations when mucoadministered. In some embodiments, non-irritative compositions and methods also exhibit no or negligible odor, taste or aftertaste.

As used herein, the term “universally,” when used in reference to non-irritative compositions and methods, refers to instances where at least 90% of the subjects to which a composition is administered experience no or negligible burning, stinging, etc. In some embodiments, the term universally includes instances where at least 95% of the subjects experience no or negligible burning, stinging, etc. In other embodiments, the term universally includes instances where 100% of the subjects experience no or negligible burning, stinging, etc.

As used herein, the term “free or essentially free of” any component refers to the presence of the component in an amount less than that which would render the component mucosally irritative. In some embodiments, the term “free or essentially free of” any component refers to less than about 3% of the component being present in a composition, e.g., a solid composition. In other embodiments, the term “free or essentially free of” any component refers to less than about 2% of the component being present in a composition, e.g., a solid composition. In other embodiments, the term “free or essentially free of” any component refers to less than about 1% of the component being present in a composition, e.g., a solid composition. In still other embodiments, the term “free or essentially free of” any component refers to less than about 0.5% of the component being present in a composition, e.g., a solid or liquid composition. In some embodiments, the term “free or essentially free of” any component refers to less than about 0.1% of the component being present in a composition, e.g., a liquid composition.

As used herein, the term “low,” when referring to plasma concentration, refers to a plasma concentration which is significantly less than a toxic concentration. Without wishing to be bound by any particular theory, it is believed that the mucoadministered compositions of the present invention are able to maintain a concentration in the plasma which is effective for treatment as described in more detail herein, without having toxic side effects typically associated with higher concentrations, e.g., liver toxicity. For example, in some embodiments, low plasma concentration refers to a concentration at least 25% less than a toxic concentration. In some embodiments, low plasma concentration refers to a concentration at least 30% less than a toxic concentration. In some embodiments, low plasma concentration refers to a concentration at least 35% less than a toxic concentration. In some embodiments, low plasma concentration refers to a concentration at least 50% less than a toxic concentration. In some embodiments, low plasma concentration refers to a concentration at least 65% less than a toxic concentration.

It is to be noted that the singular forms “a,” “an,” and “the” as used herein include “at least one” and “one or more” unless stated otherwise. Thus, for example, reference to “a pharmacologically acceptable carrier” includes mixtures of two or more carriers as well as a single carrier, and the like.

“Treatment”, or “treating” as used herein, is defined as the application or administration of a therapeutic agent to a subject who has a disorder, e.g., chronic non-invasive fungus-induced rhinosinusitis as described herein, with the purpose to cure, heal, alleviate, delay, relieve, alter, remedy, ameliorate, improve or affect the disease or disorder, or symptoms of the disease or disorder. The term “treatment” or “treating” is also used herein in the context of administering agents prophylactically. The term “effective dose” or “effective dosage” is defined as an amount sufficient to achieve or at least partially achieve the desired effect. The term “therapeutically effective dose” is defined as an amount sufficient to cure or at least partially arrest the disease and its complications in a subject already suffering from the disease.

The term “subject,” as used herein, refers to animals such as mammals, including, but not limited to, humans, primates, cows, sheep, goats, horses, pigs, dogs, cats, rabbits, guinea pigs, rats, mice or other bovine, ovine, equine, canine, feline, rodent or murine species.

For the purpose of this invention, the term “mucositis” as used herein refers to inflammation of a mucus membrane. A non-invasive fungus-induced mucositis refers to an inflammation of any mucosal tissue induced by a non-invasive fungal organism. Examples of mucosal tissue include, without limitation, the mucosa of the mouth, gut, nasal passages, paranasal sinuses, airways of the lung, trachea, middle ear, eustachian tube, vagina, and urethra. Typical inflammations of the mucous membranes include, but are not limited to, chronic non-invasive fungus-induced rhinosinusitis, chronic otitis media, chronic colitis, and Crohn's disease and chronic asthma symptoms. The term “non-invasive fungus-induced rhinosinusitis” includes any nasal-paranasal mucositis condition having a non-invasive fungal etiology. The term “chronic” as used herein refers to afflictions present for at least three months. It is to be understood that afflictions that are treated as described herein and become asymptomatic can be classified as chronic. Thus, chronic afflictions can be symptomatic or asymptomatic. As used herein, the term “mucoadministration” refers to any type of administration that places an administered agent in contact with mucus. In general, an inflammation of a mucosal tissue (e.g., chronic non-invasive fungus-induced rhinosinusitis) can be determined using methods described, e.g. in U.S. Pat. No. 6,555,566, the entire contents of which are hereby incorporated by this reference.

Numerous values and ranges are recited in connection with various embodiments of the present invention, e.g., amount of amphotericin B. It is to be understood that all values and ranges which fall between the values and ranges listed are intended to be encompassed by the present invention unless stated otherwise.

### Compositions of the Present Invention

The present invention is based, at least in part, on solid formulations that can be reconstituted into a liquid composition immediately prior to use. Some conventional liquid compositions of amphotericin B include components (e.g., propylene glycol, sodium metabisulfate, carboxymethylcellulose sodium, etc.) which act to maintain the stability of the composition and the efficacy of the amphotericin B. Without wishing to be bound by any particular theory, it is believed that solid formulations of the present invention can remain stable and maintain their efficacy for longer than conventional liquid compositions. Water can then be added immediately prior to use to form a liquid composition suitable for mucoadministration. The present invention is also based, at least in part, on the discovery that agents typically used to aid in dissolution of active ingredients may not be necessary in mucoadministered formulations. Some conventional liquid compositions of amphotericin B, e.g., FUNGIZONE, include components (e.g., deoxycholate salts) which act to increase the solubility of the active in liquid, e.g., distilled water. Accordingly, again without wishing to be bound by any particular theory, it is believed that the formulations of the present invention can be used in the treatment of non-invasive fungus induced mucositis without the necessity of an agent which aids dissolution. The present invention is also based, at least in part, on the discovery of formulations for mucoadministration that are not irritating to the mucosa. Without wishing to be bound by any particular theory, it is believed that the removal of certain agents, e.g. agents conventionally used to maintain the stability of a composition and the efficacy and solubility of the amphotericin B also removes many of the irritative properties of the formulation. Accordingly, in some aspects, the present invention provides a composition for mucoadministration including a mucosally non-irritative mixture of amphotericin B and a pharmaceutically acceptable carrier. In some embodiments, the mixture is a universally mucosally non-irritative mixture.

Compositions of the present invention generally include a pharmaceutically acceptable carrier. The pharmaceutically acceptable carrier can be a solid vehicle. For example, powders, capsules or tablets can contain amphotericin B in a form suitable for dissolution and subsequent non-irritative mucoadministration. In some embodiments, the composition is a powder. Examples of pharmaceutically acceptable solid vehicles include, but are not limited to, gelatin, starch, sugar, or bentonite.

In some embodiments, e.g. where the composition is in solid form, the amphotericin B is present in an amount of between about 0.27% and about 0.50% by weight of the composition. For example, the amphotericin B can be present in an amount of about 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49% or 0.50%. Additional amounts of amphotericin B are also suitable for the compositions of the present invention, provided that the formulation is non-irritative and not toxic.

In some embodiments, the pharmaceutically acceptable carrier includes buffering agents. The term “buffering agent” refers to one or more components which are added to a composition in order to adjust or maintain the pH of the composition. Suitable buffering agents are known to the skilled artisan and include, but are not limited to phosphates, carbonates, borates, lactates, acetates, and citrates, and combinations thereof, particularly alkali metal or alkaline metal salts of these agents. It is to be understood that buffering agents useful for the present invention are non-irritative. In some embodiments, the buffering agents are sodium phosphate buffering agents.

In some embodiments, the pharmaceutically acceptable carrier includes sodium phosphate dibasic and/or sodium phosphate monobasic. In some embodiments, e.g., where the composition is in solid form, the sodium phosphate dibasic is present in an amount of between about 45% and about 70% by weight; e.g., about 45%, 50%. 55%, 60%, 65%, or 70%. In some embodiments, e.g. where the composition is in solid form, the sodium phosphate monobasic is present in an amount of between about 30% and about 55%, e.g. about 30%, 35%, 40%, 45%, 50% or 55%. Additional amounts of sodium phosphate dibasic and/or sodium phosphate monobasic are also suitable for the compositions of the present invention, provided that the formulation is non-irritative and not toxic. In some embodiments, the compositions of the present invention include sodium phosphate dibasic in an amount of about 62%. In some embodiments, the compositions of the present invention include sodium phosphate monobasic in an amount of about 37%.

As discussed above, in some embodiments, the pharmaceutically acceptable carrier is a buffering agent or a mixture of buffering agents. In some embodiments, the pharmaceutically acceptable carrier is only buffering agent or a mixture of buffering agents. In some embodiments, the pharmaceutically acceptable carrier includes a buffering agent or a mixture of buffering agents and sterile water. In some embodiments, the pharmaceutically acceptable carrier includes only a buffering agent or a mixture of buffering agents and sterile water.

In some aspects, the present invention provides compositions for mucoadministration that include between about 0.27% and about 0.50% by weight amphotericin B; between about 60% and about 65% by weight sodium phosphate dibasic; and between about 35% and about 40% by weight sodium phosphate monobasic.

In some embodiments, e.g., where the composition is in solid form, the solid form is suitable for incorporation into a solution or suspension. For example, in some embodiments, water (e.g., sterile water) is added to the composition of the present invention to form a solution or suspension. Such a solution or suspension would be suitable for non-irritative mucoadministration.

Compositions of the present invention generally include a pharmaceutically acceptable carrier. The pharmaceutically acceptable carrier can be an aqueous vehicle, e.g., any liquid solution capable of dissolving an antifungal agent and is not toxic to the particular individual receiving the formulation. Examples of pharmaceutically acceptable aqueous vehicles include, but are not limited to, saline, water, and acetic acid. Typically, pharmaceutically acceptable aqueous vehicles are sterile. In some embodiments, the pharmaceutically acceptable carrier includes sterile water. It is to be understood that additional aqueous vehicles are also suitable for the compositions of the present invention, provided that they are non-irritative and not toxic.

Accordingly, in some embodiments, the composition is a liquid. For example, in some embodiments, the composition includes water, e.g., sterile water. In other embodiments, the composition includes saline. Typically, the saline or water used in amphotericin B formulations of the present invention is sterile. In some aspects, the present invention provides compositions which include amphotericin B, sodium phosphate dibasic, sodium phosphate monobasic, and at least 96.25% by weight water, e.g., sterile water. In some embodiments, compositions of the present invention include at least 96.50% by weight water. In some embodiments, compositions of the present invention include at least 96.75% by weight water. In some embodiments, compositions of the present invention include at least 97.00% by weight water. In some embodiments, compositions of the present invention include at least 97.25% by weight water. In some embodiments, e.g., where the composition is in liquid form, the amphotericin B is present in an amount of about 0.01% by weight of the total composition. In some embodiments, e.g., where the composition is in liquid form, the sodium phosphate dibasic is present in an amount of about 1.59% by weight of the total composition. In some embodiments, e.g., where the composition is in liquid form, the sodium phosphate monobasic is present in an amount of amount of about 0.96% by weight of the total composition. In some embodiments, e.g., where the composition is in liquid form, water is present in an amount of amount of about 97.44% by weight of the total composition.

In some aspects, the present invention provides compositions for mucoadministration that include amphotericin B; sodium phosphate dibasic; sodium phosphate monobasic; and at least about 96.25% by weight water, e.g., compositions that include about 0.01% by weight amphotericin B; about 1.59% by weight sodium phosphate dibasic; about 0.96% by weight sodium phosphate monobasic; and about 97.44% by weight water.

In some aspects, the compositions of the present invention consist essentially of amphotericin B, sodium phosphate dibasic, and sodium phosphate monobasic. In some aspects, the compositions of the present invention consist essentially of amphotericin B, sodium phosphate dibasic, sodium phosphate monobasic and water.

A formulation containing an antifungal agent can be in any form provided the formulation can be non-irritatively mucoadministered to a mammal in an amount, at a frequency, and for a duration effective to prevent, reduce, or eliminate a non-invasive fungus-induced mucositis. For example, a formulation within the scope of the invention can be in the form of a solid, liquid, and/or aerosol including, without limitation, powders, crystalline substances, gels, pastes, ointments, salves, creams, solutions, suspensions, partial liquids, sprays, nebulae, mists, atomized vapors, tinctures, pills, capsules, tablets, and gelcaps.

In some embodiments, e.g., where multiple fungal organisms or fungal organisms which are more resistant to amphotericin B are present in the mucous, the compositions and methods of the present invention include amphotericin B and one or more additional ingredients. Additional ingredients include, but are not limited to, additional antifungal agents, steroids, mucolytic agents, antibacterial agents, anti-inflammatory agents, immunosuppressants, dilators, vaso-constrictors, decongestants, leukotriene inhibitors, anti-cholinergics, anti-histamines, therapeutic compounds, compounds known to be effective for inhibiting the gag reflex of a mammal, and combinations thereof.

In some embodiments, the compositions of the present invention are free or essentially free of components that may be irritative to the mucosa, e.g., the nasal-paranasal mucosa. In some embodiments, the compositions of the present invention are free or essentially free of solvent, e.g., propylene glycol. In some embodiments, the compositions of the present invention are free or essentially free of antioxidants, e.g., sodium metabisulfate. In some embodiments, the compositions of the present invention are free or essentially free of a thickening or suspending agent, e.g., carboxymethylcellulose sodium. In still other embodiments, the compositions of the present invention are free or essentially free of antimicrobials, e.g., methylparaben and/or propylparaben. In yet other embodiments, the compositions of the present invention are free or essentially free of bile salts and/or emulsifiers, e.g., deoxycholate salts such as sodium deoxycholate. In some embodiments, compositions of the present invention are free or essentially free of combinations or mixtures of one or more solvents (e.g., propylene glycol), antioxidants (e.g., sodium metabisulfate), thickening or suspending agents (e.g., carboxymethylcellulose sodium), antimicrobials (e.g., methylparaben and/or propylparaben), bile salts and/or emulsifiers (e.g., deoxycholate salts).

In some embodiments, the compositions of the present invention are stable over a desired period of time. For example, in some embodiments, the compositions of the present invention are at least 95% stable over 3 months. In some embodiments, the compositions of the present invention are at least 95% stable over 6 months. In some embodiments, the compositions of the present invention are at least 95% stable over 9 months. In some embodiments, the compositions of the present invention are at least 95% stable over 12 months. In some embodiments, the compositions of the present invention are at least 90% stable over 18 months. In some embodiments, the compositions of the present invention are at least 95% stable over 18 months. In some embodiments, the compositions of the present invention are at least 99% stable over 18 months. It is to be understood that the stability of the compositions of the present invention may be enhanced by storage and/or preparation under an inert (e.g., nitrogen) atmosphere. Accordingly, in some embodiments, the compositions of the present invention are at least about 10% more stable under a nitrogen atmosphere than under an oxygen atmosphere. In some embodiments, the compositions of the present invention are at least about 15% more stable under a nitrogen atmosphere than under an oxygen atmosphere. In some embodiments, the compositions of the present invention are at least about 20% more stable under a nitrogen atmosphere than under an oxygen atmosphere.

In some embodiments, the compositions of the present invention provide a low plasma concentration of antifungal agent. For example, in some embodiments, the plasma concentration does not exceed about 25 ng/mL when administered in a dosage of about 0.11 mg/kg per day. In some embodiments, the plasma concentration does not exceed about 20 ng/mL when administered in a dosage of about 0.11 mg/kg per day. In some embodiments, the plasma concentration does not exceed about 15 ng/mL when administered in a dosage of about 0.11 mg/kg per day. In some embodiments, the plasma concentration does not exceed about 10 ng/ml, when administered in a dosage of about 0.11 mg/kg per day.

In some embodiments, the compositions of the present invention include antifungal agents other than amphotericin B. Suitable antifungal agents include, but are not limited to, flucytosine, ketoconazole, miconazole, itraconazole, fluconazole, griseofulvin, clotrimazole, econazole, terconazole, butoconazole, oxiconazole, sulconazole, saperconazole, voriconazole, ciclopirox olamine, haloprogin, tolnaftate, naftifine, terbinafine hydrochloride, morpholines, nystatin, natamycin, butenafine, undecylenic acid, Whitefield's ointment, propionic acid, and caprylic acid. For example, the compositions of the present invention can include a non-irritative mixture of itraconazole and a pharmaceutically acceptable carrier.

In other embodiments, the antifungal agent of the composition used in the present invention includes at least one agent selected from the group consisting of: methyl and propyl parabens, sodium benzoate, benzyl alcohol, potassium sorbate, sodium metabisulfite, thimerasol, hydrogen peroxide, sodium perborate, polyquad, polyhexamethylene, sodium silver chloride, polyquaternium-1, chlorobutanol, benzylalkonium chloride or quaternary ammonium salts. Quaternary ammonium salts include compounds of the following formula:

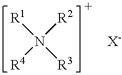

wherein N has a valency of 5;
R¹, R², R³, R⁴ are the same or different and are independently chosen from H, an alkyl group, an alkoxy group, a cycloalkyl group, a heterocycloalkyl group, an aryl group, a heteroaryl group, an acyl group, or a thioacyl group; and
X is an anion, preferably a halogen.

In a further embodiment, the quaternary ammonium salt is cetylpyridinium chloride.

In some embodiments, the compositions of the present invention further include one or more polysaccharide degrading enzymes. As used herein, the term “polysaccharide degrading enzyme” refers to an enzyme that cleaves glycosidic bonds. Without wishing to be bound by any particular theory, it is believed that such an enzyme would cleave the glycosidic bonds of polysaccharides present in mucus and, thereby aid in breaking up thick secretions, e.g., by reducing the viscosity of mucus. Examples of a polysaccharide degrading enzyme include, but are not limited to, β-glucosidase, pullulanase, neuraminidase and hyaluronidase. In a particular embodiment, the polysaccharide degrading enzyme is hyaluronidase.

Accordingly, in some embodiments, the present invention provides co-administration of a mucosally non-irritative antifungal formulation and a polysaccharide degrading enzyme. The polysaccharide degrading enzyme can be, e.g., any polysaccharide degrading enzyme listed above. In one embodiment, the polysaccharide degrading enzyme is hyaluronidase.

### Dosages, Frequency and Duration

In some embodiments, the compositions of the present invention include an amount of amphotericin B or other antifungal agent which can be mucoadministered to a subject at a frequency and for a duration effective to treat or prevent non-invasive fungus-induced mucositis. An effective amount of amphotericin B or other antifungal or composition including amphotericin B can be any amount that reduces, prevents, or eliminates non-invasive fungus-induced mucositis upon mucoadministration in a subject without producing significant toxicity to the subject. Typically, an effective amount can be any amount greater than or equal to the minimum inhibitory concentration (MIC) for a fungal organism or isolate present within a particular individual's mucus that does not induce significant toxicity to the individual upon mucoadministration. The effective amount can vary depending upon the specific fungal organism or isolate since certain organisms and isolates are more or less susceptible to particular antifungal agents. Such effective amounts can be determined using commonly available or easily ascertainable information involving antifungal effectiveness concentrations, animal toxicity concentrations, and tissue permeability rates. Using the information provided herein, such effective amounts also can be determined by routine experimentation in vitro or in vivo. For example, a patient having a non-invasive fungus-induced mucositis condition can receive direct mucoadministration of an antifungal agent in an amount close to the MIC calculated from in vitro analysis. If the patient fails to respond, then the amount can be increased by, for example, ten fold. After receiving this higher concentration, the patient can be monitored for both responsiveness to the treatment and toxicity symptoms, and adjustments made accordingly.

Various other factors can influence the actual effective amount used for a particular application. For example, the frequency of mucoadministration, duration of treatment, combination of other antifungal agents, site of administration, degree of inflammation, and the anatomical configuration of the treated area may require an increase or decrease in the actual effective amount mucoadministered.

For amphotericin B, an effective amount can be about 0.01 ng to about 1000 mg per kg of body weight of the mammal per administration when mucoadministered. When used as a nasal irrigation solution, an effective amount can be a volume of about 0.01 mL to about 1 liter per nostril per administration of a solution containing about 0.01 mg of amphotericin B per liter to about 1000 mg of amphotericin B per liter. For example, compositions of the present invention can include 0.01 mg, 0.10 mg, 0.25 mg, 0.50 mg, 0.75 mg, 1.0 mg, 5 mg, 10 mg, 25 mg, 50 mg, 75 mg, 100 mg, 250 mg, 500 mg, 750 mg, or 1000 mg of amphotericin B per liter of liquid carrier. Exemplary volumes of such solutions to be administered can include 0.01 mL, 0.10 mL, 0.25 mL, 0.50 mL, 0.75 ml, 1.0 mL, 5 mL, 10 mL, 25 mL, 50 mL, 75 mL, 100 mL, 250 mL, 500 mL, 750 mL, or 1 L. The skilled artisan would be able to determine the volume necessary to deliver an effective amount based upon the concentration of amphotericin B in the formulation. For example, in some embodiments, the composition includes between about 50 μg and about 1000 μg per milliliter of sterile water, e.g., between about 100 μg and about 500 μg per milliliter of sterile water. In some embodiments, the composition includes about 100 μg of Amphotericin B per milliliter of sterile water. In other embodiments, the effective amount includes about 5 mL to about 100 mL of the composition per nostril of the subject. In still other embodiments, an effective amount comprises about 20 mL of the composition per nostril of the subject.

In some embodiments, mucoadministration is achieved via a pump spray. Accordingly, in some embodiments, mucoadministration includes from 1 to 4 pumps per nostril, e.g., 3 pumps per nostril. In some embodiments, the pump dispenses between about 50 μL and about 200 μL of the composition, e.g, about 100 μL of the composition. In other embodiments, an effective amount is 20 mL per nostril per administration (e.g., two to four times daily) of an irrigation solution containing about 100 mg of amphotericin B per liter of saline or water.

Any effective amount of amphotericin B described herein may be used provided that it is mucosally non-irratative and not toxic to the subject. The effective amount can remain constant or can be adjusted as a sliding scale or variable dose depending on the individual's response to treatment.

It is to be understood that the volume administered can be administered in one single event or in multiple events. For example, 20 mL per nostril of an irrigation solution containing about 100 mg of amphotericin B per liter of saline or water can be administered as a continuous irrigation, wash or spray of the entire 20 mL. Alternatively, administration can include two or more sequential applications of a portion of the exemplary 20 mL (e.g., 2 washes/sprays of 10 mL each, 4 washes/sprays of 5 mL each, etc.). It is also to be understood that, when a volume is administered via two or more sequential applications, each application need not be equivalent to the previous application. For example, 20 mL of a formulation can be administered via 1 wash/spray of 10 ml followed by 2 washes/sprays of 5 mL each.

By way of a further example, a nonirritative composition of the present invention having a concentration of between about 50 μg/ml (amphotericin B/liquid carrier, e.g., sterile water) and about 1000 μg/ml, e.g., between about 100 μg/ml and about 500 μg/ml, can be administered by a pump from one to four times daily (e.g., three times daily). Each pump can have a volume of between about 50 μL and about 200 μL, e.g., a volume of about 100 μL. Furthermore, each single mucoadministration event can include from one to four pumps per nostril, e.g., three pumps per nostril.

The frequency of mucoadministration can be any frequency that reduces, prevents, or eliminates non-invasive fungus-induced mucositis in a mammal without producing significant toxicity to the mammal. For example, the frequency of mucoadministration can be from about four times a day to about once a month, or more specifically, from about twice a day to about once a week. The frequency of mucoadministration can be four times a day, three times a day, two times a day, once a day, every other day, every third day, twice a week, once a week, once every two weeks, once every three weeks, or once a month. In addition, the frequency of mucoadministration can remain constant or can be variable during the duration of treatment. For example, the first three doses may occur within day one at a frequency of three times a day, but the next four doses may be administered at a frequency of twice a day, once a day, etc. As with the effective amount, various factors can influence the actual frequency of mucoadministration used for a particular application. For example, the effective amount, duration of treatment, combination of other antifungal agents, site of administration, degree of inflammation, and the anatomical configuration of the treated area may require an increase or decrease in mucoadministration frequency.

An effective duration for antifungal agent mucoadministration can be any duration that reduces, prevents, or eliminates non-invasive fungus-induced mucositis in a mammal without producing significant toxicity to the mammal. Thus, the effective duration can vary from several days to several weeks, months, or years. In general, the effective duration for the treatment of non-invasive fungus-induced mucositis can range in duration from several days to several months. Once the antifungal applications are stopped, however, non-invasive fungus-induced mucositis may return. Thus, the effective duration for the prevention of non-invasive fungus-induced mucositis can last in some cases for as long as the individual is alive. In some embodiments, an effective duration is at least 7 days. In other embodiments, an effective duration is at least 14 days. In still other embodiments, an effective duration is at least 30 days, 60 days, 90 days, 3 months, 6 months, 9 months, 1 year or more.

Multiple factors can influence the actual effective duration used for a particular treatment or prevention regimen. For example, an effective duration can vary with the frequency of amphotericin B administration, effective amount, combination of amphotericin B with other agents (e.g., other antifungal agents), site of administration, degree of inflammation, and anatomical configuration of the treated area.

It is noted that diagnostic algorithm methods can be devised to determine or reflect appropriate effective doses, durations, and frequencies without any undue experimentation.

### Methods for Treating Non-Invasive Fungus-Induced Mucositis

In some aspects, the present invention provides methods for treating non-invasive fungus-induced mucositis. The method generally includes the mucoadministration of any of the compositions provided herein to a subject. Accordingly, in some embodiments, the non-irritative compositions of the present invention are suitable for administration to the mucosa (e.g., for mucoadministration to the nasal-paranasal cavities). In some embodiments, the composition of the present invention is administered in an amount, at a frequency, and for a duration effective to reduce or eliminate the non-invasive fungus-induced mucositis.

In general, most, if not all, individuals have fungal organisms living in their mucus. Normally, most individuals tolerate these non-invasive organisms and live normal disease-free lives. Some individuals do not tolerate these fungal organisms and begin to mount an immune response against them. As the immune response progresses, eosinophils accumulate within the local tissue. This accumulation of eosinophils can contribute to the formation of obstructive tissue masses (e.g., polyps and polypoid structures) as well as the transmigration of activated eosinophils from the tissue (inside the body) to the mucus (outside the body). These obstructive tissue masses appear to prevent normal cavity clearance and thus can facilitate additional fungal growth. Once eosinophils are within the mucus, they can release the contents of their granules presumably upon the activation of surface Fc receptors. Eosinophil granules contain many toxic molecules such as eosinophil cationie protein (ECP), eosinophil peroxidase (EPO), and major basic protein (MBP). Upon release, these toxic molecules can damage both the targeted foreign microorganisms (e.g. fungus) as well as self tissues. The degree of damage caused by eosinophil accumulation and eosinophil degranulation varies significantly from slight inflammatory pain and discomfort to major structural abnormalities such as tissue and bone destruction and the formation of polyps, polypoid structures, and other tumors. Once self tissues are damaged, the individual can have an increased susceptibility to bacterial infections as well. Thus, the characteristic inflammatory responses, resulting damages, and resulting bacterial infections observed within most, if not all, chronic rhinosinusitis patients are actually triggered by non-invasive fungal organisms. In some aspects, the present invention provides methods and compositions to reduce the amount of eosinophil and/or MBP in the mucosa of a subject. The methods and compositions can be any of those described herein.

It is noted that fungal organisms may be observed within the tissue under extreme mucositis conditions of tissue and bone destruction simply because the barrier (i.e., epithelium) between the inside and outside of the body has been destroyed or damaged. In these situations, the mere observed presence of a small number of fungal organisms within a localized area of tissue damage does not deter from the fact that the affliction is a non-invasive fungus-induced mucositis and not an infection.

Any fungal organism living in the mucus of a mammal can be a non-invasive fungal organism that is capable of inducing mucositis since it is the mere presence of the organism in an intolerant individual's mucus that causes inflammation. Exemplary fungal organisms include, but are not limited to, Absidia, Aspergillus flavus, Aspergillus filmigatus, Aspergillus glaucus, Aspergillus nidulans, Aspergillus versicolor, Alternaria, Basidiobolus, Bipolaris, Candida albicans, Candida lypolytica, Candida parapsilosis, Cladosporium, Conidiobolus, Cunninahamella, Curvularia, Dreschlera, Exserohilum, Fusarium, Malbranchia, Paecilomyces, Penicillium, Pseudallescheria, Rhizopus, Schizophylum, Sporothrix, Acremonium, Arachniotus citrinus, Aurobasidioum, Beauveria, Chaetomium, Chryosporium, Epicoccum, Exophilia jeanselmei, Geotrichum, Oidiodendron, Phoma, Pithomyces, Rhinocladiella, Rhodoturula, Sagrahamala, Scolehasidium, Scopulariopsis, Ustilago, Trichoderma, and Zygomycete. Additional fungal organisms that can be non-invasive fungal organisms capable of inducing a non-invasive fungus-induced mucositis can be found in most taxonomic mycology text books.

As described herein, the present invention provides non-irritative methods and compositions that reduce the presence of fungal organisms within mucus to a level and for a period of time such that the characteristic inflammatory responses and resulting damages associated with mucositis are stopped, treated, or prevented.

Mucosa from any mucosal tissue can be treated with the compositions of the present invention. Examples of mucosal tissue include, but are not limited to, the mucosa of the mouth, gut, nasal passages, paranasal sinuses, airways of the lung, trachea, middle ear, eustachian tube, vagina, and urethra. In certain embodiments, the mucosa treated in the present invention is from the nasal passages and/or paranasal sinuses.

In some embodiments, the present invention provides methods for non-irritatively mucoadministering any of the compositions described herein to the nasal-paranasal cavities. Mucosal tissue (mucosa) lines both the nasal cavity and the paranasal sinuses, and generally comprises an epithelial layer, connective tissue, and mucus glands. A layer of mucus normally covers the mucosa. Mucus secreted from mucosa serves to trap particles and to prevent dehydration of the nasal and paranasal tissues that are otherwise exposed to air. The mucus is normally transported by cilia toward the nasopharynx and then swallowed.

The mucoadministration of an agent, e.g., to the nasal-paranasal anatomies, can be any type of administration that places the agent in contact with mucus, e.g., direct or indirect mucoadministration. In some embodiments, the mucoadministration of a composition of the present invention is direct mucoadministration. Direct mucoadministration to the nasal-paranasal anatomies can include, without limitation, nasal irrigations, nasal sprays, nasal inhalations, and nasal packs with, for example, saturated gauze provided the administered agent contacts nasal-paranasal mucus prior to crossing epithelium. In addition, injections into the nasal-paranasal cavities using, for example, a needle or catheter tube is considered a direct mucoadministration provided the administered agent contacts nasal-paranasal mucus after leaving the needle or catheter tube and prior to crossing epithelium. Any device can be used to directly mucoadminister an agent to the nasal-paranasal anatomy including, without limitation, a syringe, bulb, inhaler, canister, spray can, nebulizer, and mask. For example, a 20 mL bulb can be used to irrigate the nasal-paranasal anatomy with a liquid form of a formulation containing an antifungal agent. Such a liquid form of a formulation can be stored at −20° C., 0° C., or room temperature. If stored below room temperature, the formulation typically is warmed prior to application to the nasal/paranasal cavities.

In some embodiments, the subject had a nasal surgery before said mucoadministration. In other embodiments, the subject was nasal surgery-free before said mucoadministration. The compositions and methods of the present invention are useful for both subjects who have undergone nasal surgery as well as subjects who have never had nasal surgery. In some embodiments, mucoadministration begins during a period noncoincident with an intraoperative period, said intraoperative period being the time during a nasal surgery.

The compositions of the present invention are useful for the treatment of any non-invasive fungus-induced mucositis conditions. Typical such conditions generally involve inflammations of the mucous membranes which include, but are not limited to, chronic non-invasive fungus-induced rhinosinusitis, chronic otitis media, chronic colitis, and Crohn's disease and chronic asthma symptoms.

In some embodiments, the present invention provides methods for treating non-invasive fungus-induced rhinosinusitis. Individuals suffering from rhinosinusitis can be identified using methods commonly known in the art. Symptoms of rhinosinusitis include, without limitation, nasal airway obstruction, loss of smell, facial pain, head ache, post nasal drip, and rhinorrhea. Upon examination, the presence of thick mucus or the visual identification of nasal or paranasal obstruction with mucus or polyps often indicates a rhinosinusitis condition. The presence of nasal polyps is not a risk factor for rhinosinusitis, but rather an end stage of chronic inflammation. Nasal polyps are outgrowths from the nasal-paranasal mucosa that are typically smooth, gelatinous, semitranslucent, round or pear shaped, and pale. The mass of a nasal polyp is composed mainly of edematous fluid with sparse fibrous cells and a few mucous glands. The surface epithelium of nasal and paranasal polyps generally reveals squamous metaplasia. Eosinophils are usually present in polyps in moderate to large numbers, and it is now known that nasal polyp fluid contains greater than normal concentrations of IgA, IgE, IgG, and IgM antibodies as well as abnormally high concentrations of IL-5, a cytokine that contributes to eosinophil activation and survival.

Any individual that had a previous episode of rhinosinusitis is at risk for developing non-invasive fungus-induced rhinosinusitis. In addition, elderly individuals as well as individuals having cystic fibrosis, asthma, and a family history of nasal problems or allergies can be at risk for developing non-invasive fungus-induced rhinosinusitis. Further, individuals that are exposed to significant levels of allergens (e.g., fungus spores, pollen, and chemicals) can be at risk for developing non-invasive fungus-induced rhinosinusitis. Accordingly, in some embodiments, the present invention provides compositions and methods for non-irritatively treating a subject at risk for developing non-invasive fungus-induced mucositis.

Other treatments can be used in combination with a formulation containing an antifungal agent to help enhance the treatment or prevention of non-invasive fungus-induced mucositis conditions. Such additional treatments can include, without limitation, surgeries and the administration of a second formulation. Surgeries can include, without limitation, the removal of polypoid growths or other tumors, the physical opening of a cavity, and the insertion of catheter tubes and the like. A second formulation can include, without limitation, antifungal agents, mucolytic agents, antibacterial agents, anti-inflammatory agents, immunosuppressants, dilators, vaso-constrictors, decongestants, steroids, anti-cholinergics, leukotriene inhibitors, anti-histamines, therapeutic compounds, and combinations thereof. In addition, this second formulation can be administered to a mammal by any route. For example, oral, intraperitoneal, intradermal, intravenous, subcutaneous, intramuscular, topical, intranasal, and intrabronchial administration can be used to deliver a second formulation to a mammal.

In some embodiments, the present invention also provides a method for treating and preventing asthma using compositions as described herein. Asthma can be characterized by a paradoxical narrowing of the bronchi (lung passageways) such that breathing becomes difficult. Individuals suffering from asthma can exhibit symptoms such as wheezing, difficulty breathing (particularly exhaling air), dyspnea, and tightness in the chest. Factors that can exacerbate asthma include rapid changes in temperature or humidity, allergies, upper respiratory infections, exercise, stress, and smoking. Individuals suffering from asthma can be identified using any of the known methods in the art.

Further, individuals at risk for developing chronic asthma can be prophylactically treated by mucoadministering an antifungal agent to at least a portion of the airways in an amount, at a frequency, and for a duration effective to prevent asthma symptoms. Again, such prophylactic treatments can be similar to the methods and materials described herein for the prophylactic treatment of non-invasive fungus-induced rhinosinusitis.

The invention will be further described in the following examples, which are not meant to limit the scope of the invention in any way.

### EXAMPLES

### Example 1

### Preparation of an Exemplary Amphotericin B Solution of the Present Invention

To minimize concerns regarding stability, the drug product formulation has been modified. Original formulations used in clinical studies reported in the literature generally included an aqueous suspension of a formulation of amphotericin B, based for example, on oral FUNGIZONE (Bristol-Myers Squibb). A suspension of FUNGIZONE includes not only amphotericin B, but also sodium phosphate dibasic, sodium phosphate monobasic, and sodium desoxycholate. Conventional amphotericin B formulations may also include, for example, carboxymethylcellulose sodium, sodium metabisulfite, propylene glycol, methylparaben, and propylparaben,

A composition of the present invention is produced by mixing amphotericin B, sodium phosphate dibasic, and sodium phosphate monobasic, e.g., in the ratios provided below. For example 6.0 mg of amphotericin B, 956.5 mg sodium phosphate dibasic, and 577.5 mg sodium phosphate monobasic, can be mixed and placed into a foil laminate sachet. This mixture can then be reconstituted with sterile water immediately prior to use. For each sachet that contains 1.54 grams of solid, 58.5 ml sterile water can be added to provide a composition with a final amphotericin B concentration of 100 μg/ml. One Amphotericin B composition formulation (“Suspension”) and an exemplary composition of the present invention made from the powder formulation (“Powder”) of the present invention are listed below. Percentages are listed for both the dry formulation (prior to the addition of sterile water) and the liquid formulations.

| | | | % w/w | |
|---|---|---|---|---|
| | % w/w | | (dry formulation) | |
| | Suspen- | | Suspen- | |
| Ingredient | sion | Powder | sion | Powder |
| Amphotericin B, USP | 0.01 | 0.01 | 0.26% | 0.39% |
| Sodium Phosphate Dibasic, | 1.59 | 1.59 | 41.41% | 62.11% |
| Anhydrous, USP | | | | |
| Sodium Phosphate Monobasic, | 0.96 | 0.96 | 25.00% | 37.50% |
| Anhydrous, USP | | | | |
| Carboxymethylcellulose | 0.13 | | 3.39% | |
| Sodium | | | | |
| Sodium Metabisulfite | 0.15 | | 3.91% | |
| Propylene Glycol | 0.88 | | 22.92% | |
| Methylparaben | 0.10 | | 2.60% | |
| Propylparaben | 0.02 | | 0.52% | |
| Sterile Water | 96.16 | 97.44 | — | — |

As a further comparison, a conventional suspension of FUNGIZONE in water typically includes 50 mg Amphotericin B, 41 mg sodium desoxycholate and 20.2 mg sodium phosphates as a buffer.

### Example 2

### Feasibility of Repeated Nasal Lavage Administrations

A 7-day pilot study to evaluate the feasibility of repeated nasal lavage administrations of phosphate buffer, placebo and 5× drug to Gottingen mini pigs was performed.

One treatment group of two female Gottingen mini-pig swine were administered a composition formulated from the “Powder” formulation as described in Example 1, at a dose concentration of 5 times the human dose. A second group of two females were administered a placebo (sodium phosphate dibasic, sodium phosphate monobasic, calcium carbonate, Yellow #5 lake, and water). A third group of two females received the vehicle control, sodium phosphate dibasic, sodium phosphate monobasic, and water.

The powder formulation, placebo, or vehicle were administered via nasal lavage, twice per day at approximately the time each day, approximately 6 hours apart, for 7 consecutive days. The volume for the respective treatments was normalized against a standard weight of 70 kg. The volume per nare was calculated as 20 mL×[weight animal (kg)]/[70 kg].

A complete physical examination was conducted on all animals pretest. Observations for mortality, morbidity, injury, and the availability of food and water were conducted twice daily for all animals. Clinical observations were conducted daily, approximately 2 hours following the first dose. Observations were made and recorded during each dose administration to document if the animal struggled and the severity of struggling. In turn, the approximate loss of dosing material, if any, during administration was recorded. Body weights were measured and recorded prior to randomization on Day-1 and at the end of the study. Blood samples for clinical pathology evaluations were collected from all animals on Day 7 prior to the terminal necropsy. At study termination (Day 8), necropsy examinations were performed, organ weights were recorded, and selected tissues were collected and preserved.

All animals survived during the 7-day dose administration period. No effect of treatment was seen in body weight, clinical findings, clinical pathology, organ weight, or macroscopic pathology findings. In general, most animals exhibited only mild struggling during dosing and little to no loss of dosing material was noted during dose administration. As most animals became accustomed to the procedure, the level of struggle severity tended to decrease by the end of the 7-day dosing period. During the last 2 days of dosing, the animal's morning and afternoon ration of food was presented following each dose administration. This routine had a positive impact on the animal's disposition prior to and during the dose.

### Example 3

### Repeated Nasal Lavage Administrations Over a 180-Day Study

A 180-day study to evaluate the effect of repeated nasal lavage administrations of phosphate buffer, placebo and drug (1×, 5× and 10×) to Gottingen Mini-Pigs was performed.

The three treatment groups consisted of four female and four male Gottingen mini-pig swine, and were administered a composition formulated from the “Powder” formulation as described in Example 1, at dose concentrations of 1×, 5× and 10× the human dose, respectively. The powder formulation was administered via nasal lavage, twice per day at approximately the time each day, approximately 6 hours apart, for 180 consecutive days. The volume for the respective treatments was normalized against a standard weight of 70 kg. The volume per nare was calculated as 20 mL×[weight animal (kg)]/[70 kg].

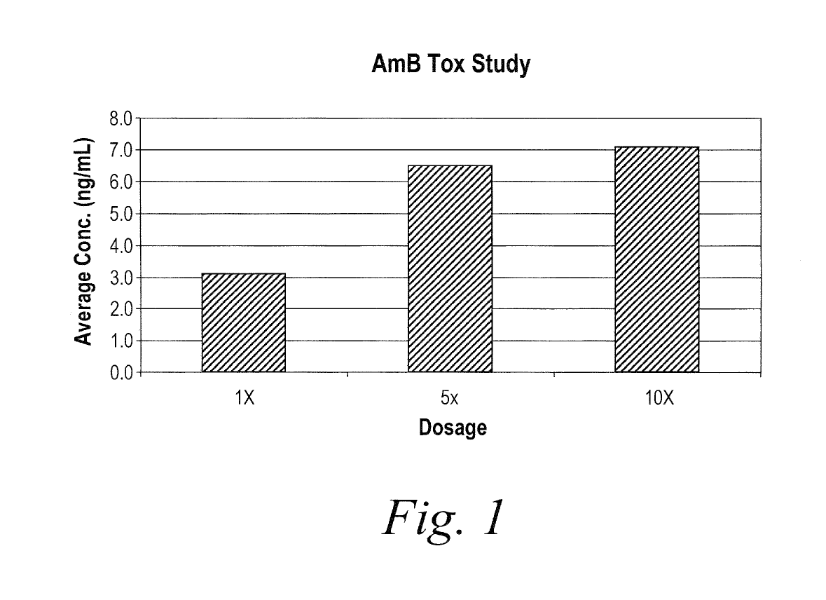

Physical examinations and clinical observations were conducted in the same manner as described above in Example 2. Blood samples for clinical pathology evaluations were collected from all animals each day at times 0, 0.1, 1, 4, 12 and 24 hours. Data is shown in FIG. 1 for the sample collected on day 135 at 24 hours.

All animals survived during the 180-day dose administration period. No effect of treatment was seen in body weight, clinical findings, clinical pathology, organ weight, or macroscopic pathology findings. Additionally, as seen in FIG. 1, the compositions used in the present example maintained a low plasma level, e.g., as shown by the sample collected on day 135. Accordingly, exemplary compositions of the present invention are able to provide low absorption of Amphotericin B upon administration, e.g., administration over an extended time period.

### Example 4

### Non-Irritatively Treating and Preventing Non-Invasive Fungus-Induced Rhinosinusitis

Rhinosinusitis patients were studied to evaluate the use of the compositions as prepared in Example 1 to treat non-invasive fungus-induced rhinosinusitis.

Diagnostic analysis showed that the patients had the following criteria prior to the study: (1) symptoms with a duration of greater than 12 weeks; (2) presence of observable disease within the nasal-paranasal anatomy as evidenced by a CT scan, (e.g. at least 5 mm mucosal thickening in at least 1 maxillary sinus at the level of the middle meatus); (3) endoscopy to exclude presence of polyps that are stage 4 in middle meatus and document presence of inflammation, such as polypoid thickening of the mucosa, discolored mucus or edema of the middle meatus or ethmoid area; and optionally (4) a history of at least 1 prior maxillary sinus surgery for CS consisting of antrostomy with or without polypectomy greater than or equal to 6 months prior to randomization. Additionally, diagnostic analysis may show the presence of allergic mucus as evidenced by histologic evaluation of a surgical specimen and/or the presence of fungal organisms within nasal-paranasal mucus as evidenced by the ability to culture fungal organisms from a mucus sample. The patients were administered about 20 mL of the solution per nostril (approximately one to three sprays per nostril), two to four times daily for at least three months. The concentration of the amphotericin B solution was 100 mg per liter of sterile water.

In addition to patient interviewing CT scan analysis visual examination, and fungal culture analysis, two types of evaluations were used to score the success of the treatment: an endoscopic evaluation and a patient symptom evaluation. These evaluations were scored as follows:

Endoscopic Evaluation Stage 0: no evidence of disease; Stage 1: polypoid changes/polyps seen by endoscopy only; Stage 2: polyps in the middle meatus; Stage 3: polyps filling the nasal cavity. Patient Symptom Evaluation Stage −2: very bad/much worse; Stage −1: bad/worse; Stage 0: baseline/no change; Stage 1: good/improved; Stage 2: very good/free of symptoms.

Analysis of the data from the blinded trial demonstrated that the exemplary compositions of the invention and the placebo provided an improvement in polyposis by endoscopy in about 50% of patients and improvement in sinus inflammation by CT scan in approximately 50% of patients. It is expected that the beneficial results of the study are due to the exemplary compositions of the invention rather than the placebo.

Analysis of the data from the blinded trial demonstrates that the amphotericin B compositions are non-irritative and can be used to effectively treat non-invasive fungus-induced rhinosinusitis. The amphotericin B composition used in the trial demonstrated a 2-4% incidence of nasal burning compared to a 15-20% incidence of burning and irritation in previous compositions.

### Example 5

### Stability of Exemplary Compositions of the Invention

The stability of the powder compositions (regular and anhydrous) of the present invention was measured via HPLC after the compositions were stored for an 18-month period under an oxygen atmosphere (regular composition only) and under a nitrogen atmosphere. Results were calculated as percent area under the HPLC curve.

The results show that the regular powder formulation was about 93% stable under nitrogen and the anhydrous powder formulation was about 98% stable under nitrogen. This is in contrast to the regular powder formulation under oxygen, which was about 74% stable. Accordingly, the data shows that exemplary compositions of the present invention exhibit about 20% greater stability under a nitrogen atmosphere than under an oxygen atmosphere.

### Prospective Example 1

### Further Evaluation of Non-Irritatively Treating and Preventing Non-Invasive Fungus-Induced Rhinosinusitis

Rhinosinusitis patients will again be studied to evaluate the use of the compositions as prepared in Example 1 to treat non-invasive fungus-induced rhinosinusitis.

It is expected that endoscopic evaluation will demonstrate most patients improving at least one stage after three months. In some cases, it is expected that patients will improve two or three stages after three months. Cases showing no evidence of disease after treatment can be confirmed by CT scans.

It is also expected that patient symptom evaluation will demonstrate most patients exhibiting stage 2 or stage 1. It is also expected that these results may correlate to the endoscopic evaluation, i.e., that patients exhibiting a stage 1 or stage 0 will show less improvement as measured by endoscopic evaluation.

The skilled artisan would understand that, should a patient not respond to the treatment, it is expected that such a patient would exhibit fungal organisms within their mucus that are known to be resistant to amphotericin B.

If patients have mucus samples collected and analyzed before and after treatment with the compositions of the present invention, it is expected that the number of different fungal species in those patients will be remarkably reduced after treatment as determined by fungal organism culturing techniques.

Patients may have had recent nasal surgery, may be using topical and systemic steroid therapy and/or may be using an antibiotic nasal irrigation in addition to the compositions of the invention. Additionally, patients may have other diseases, e.g., asthma and/or colitis.

It is expected that the results will indicate that non-irritative amphotericin B compositions of the present invention can be used effectively to treat non-invasive fungus-induced rhinosinusitis.

### Prospective Example 2

### Diminishing Eosinophilia Using an Antifungal Treatment

A patient diagnosed with non-invasive fungus-induced rhinosinusitis who has undergone sinus surgery previously will be instructed to mucoadminister amphotericin B twice a day using a composition as described in Example 1. After an extended period of time, e.g., nine months, the patient will undergo sinus surgery for further improvement. During the surgery mucosal biopsies will be collected and the eosinophil count will be compared to those obtained from biopsies collected from the patient during a surgery prior to the amphotericin B treatment.

It is expected that the eosinophil count in the mucosal biopsies from the sinuses will be diminished to less than about 5%. It is possible that the frontal sinus biopsy will not be diminished to less than 5% because it is sometimes difficult to mucoadminister a formulation to the frontal sinus due to frontal sinus obstruction. It is expected, however, that all properly treated areas will show diminished eosinophil counts.

### Prospective Example 3

### Treating and Preventing Chronic Asthma Symptoms

A patient with no history or symptoms of chronic rhinosinusitis who exhibits significant asthma symptoms will be treated with an amphotericin B formulation prepared as described in Example 1. 20 mL of the formulation will be mucoadministered in each nostril at least two times daily for an extended period of time (e.g., at least about two weeks). It is expected that the patient will report no or few episodes of shortness of breath and no wheezing during the treatment period.

Additionally, it is expected that the patient will exhibit improved pulmonary function, improved forced vital capacity (FVC) of the lung, an increased forced expiratory volume in 1 second (FEV1), improved maximal forced expiratory flow (FEFmax), and/or improved maximum voluntary ventilation (MVV). It is expected that the results will demonstrate that chronic asthma symptoms can be treated and prevented by mucoadministering the compositions of the present invention to the airways.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition for mucoadministration comprising: a mucosally non-irritative mixture of amphotericin B and a pharmaceutically acceptable carrier.

2. The composition of claim 1, wherein the mixture is a universally mucosally non-irritative mixture.

3. The composition of claim 1, wherein the pharmaceutically acceptable carrier comprises sodium phosphate dibasic and sodium phosphate monobasic.

4. The composition of claim 3 wherein the composition is a powder.

5. The composition of claim 4, wherein the amphotericin B is present in an amount of between about 0.27% and about 0.50% by weight of the total composition.

6. The composition of claim 3, further comprising water.

7. The composition of claim 6, wherein the amphotericin B is present in an amount of about 0.01% by weight of the total composition.

8. The composition of claim 1, wherein the pharmaceutically acceptable carrier comprises sodium phosphate dibasic, sodium phosphate monobasic and water.

9. The composition of claim 1, wherein the composition consists essentially of amphotericin B, sodium phosphate dibasic, sodium phosphate monobasic and water.

10. The composition of claim 1, wherein the composition is in a solid form, and wherein the solid form is suitable for incorporation into a solution or suspension.

11. The composition of claim 1, comprising:
between about 0.27% and about 0.50% by weight amphotericin B;
between about 45% and about 70% by weight sodium phosphate dibasic; and
between about 30% and about 55% by weight sodium phosphate monobasic.

12. The composition of claim 11, wherein the composition is a powder.

13. The composition of claim 12, wherein the composition is suitable for incorporation into a solution or suspension.

14. The composition of claim 1, comprising:
amphotericin B;
sodium phosphate dibasic;
sodium phosphate monobasic; and
at least about 96.25% by weight water.

15. The composition of claim 14, wherein the composition comprises:
about 0.01% by weight amphotericin B;
about 1.59% by weight sodium phosphate dibasic;
about 0.96% by weight sodium phosphate monobasic; and
about 97.44% by weight water.

16. The composition of claim 1, wherein the composition is free or essentially free of one or more of the following: propylene glycol and/or sodium metabisulfate, carboxymethylcellulose sodium, methylparaben, propylparaben, sodium desoxycholate.

17. The composition of claim 1, wherein the composition further comprises a polysaccharide degrading enzyme.

18. The composition of claim 1, wherein the composition is at least about 90% stable for up to 18 months under a nitrogen atmosphere.

19. The composition of claim 1, wherein the composition is at least about 95% stable for up to 18 months under a nitrogen atmosphere.

20. The composition of claim 1, wherein the composition is at least about 20% more stable under a nitrogen atmosphere than under an oxygen atmosphere.

21. A method of treating a subject having non-invasive fungus-induced mucositis, comprising mucoadministering an effective amount of a composition to the subject, wherein the composition comprises a mucosally non-irritative mixture of amphotericin B and a pharmaceutically acceptable carrier.

22. The method of claim 21, wherein the composition comprises between about 50 μg to about 1000 μg of amphotericin B per milliliter of sterile water.

23. The method of claim 21, wherein the composition comprises between about 100 μg to about 500 μg of amphotericin B per milliliter of sterile water.

24. The method of claim 21, wherein the composition provides a low maximum plasma concentration.

25. The method of claim 21, wherein polyposis is improved in the subject.

26. The method of claim 21, wherein sinus inflammation is improved in the subject.

27. The method of claim 21, wherein the composition comprises about 100 μg of amphotericin B per milliliter of aqueous carrier and wherein 20 ml of the composition is administered in each nostril twice daily.

28. A method for reducing eosinophil in a subject comprising: non-irritatively mucoadministering a composition comprising an effective amount of amphotericin B and a pharmaceutically acceptable carrier.

29. A method for reducing the amount of major basic protein in the mucosa of a subject comprising: non-irritatively mucoadministering a composition comprising an effective amount of amphotericin B and a pharmaceutically acceptable carrier.
